(19) United States
(12) Patent Application Publication
(10) Pub. No.: US 2010/0021967 A1
(43) Pub. Date: Jan. 28, 2010

(54) Alpha Factor Signal Peptide For Producing a Polypeptide

(75) Inventor: Henriette Draborg, Frederikssund (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/445,495

(22) PCT Filed: Oct. 23, 2007
(86) PCT No.: PCT/EP2007/061344

§ 371 Date: Apr. 14, 2009

(30) Foreign Application Priority Data
Oct. 24, 2006 (DK) PA 2006 01375

(51) Int. Cl. C12P 21/00; C07K 7/08; C07H 21/00; C12N 15/63; C12N 1/15

(52) U.S. Cl. 435/69.4; 435/69.1; 435/69.6; 530/326; 536/23.1; 435/320.1; 435/254.11

(57) ABSTRACT

The present invention relates to a method for producing a polypeptide comprising using a variant alpha factor signal peptide having a substitution in position 9 resulting in a substitution of an A to either T, S, H, I, F, E, or G. The invention further relate to nucleic acid constructs comprising a first nucleotide sequence encoding the variant signal peptide and a second nucleotide sequence encoding a polypeptide which is foreign to the first nucleotide sequence. Furthermore, it also relates to expression vectors and host cells comprising said nuclei acid construct.

### REFERENCE TO A SEQUENCE LISTING

This application contains a Sequence Listing in computer readable form. The computer readable form is incorporated herein by reference.

### FIELD OF THE INVENTION

The present invention relates to methods for producing and secreting a polypeptide and to nucleic acid constructs comprising a first and a second nucleotide sequence encoding a signal peptide and the polypeptide and expression vectors and host cells comprising said nucleic acid construct.

### BACKGROUND OF THE INVENTION

The recombinant production of a heterologous protein in a fungal host cell, particularly a filamentous fungal cell such as Aspergillus or a yeast cell such as Saccharomyces, may provide for a more desirable vehicle for producing the protein in commercially relevant quantities.

Recombinant production of a heterologous protein is generally accomplished by constructing an expression cassette in which the DNA coding for the protein is placed under the expression control of a promoter, excised from a regulated gene, suitable for the host cell. The expression cassette is introduced into the host cell. Production of the heterologous protein is then achieved by culturing the transformed host cell under inducing conditions necessary for the proper functioning of the promoter contained on the expression cassette.

Improvement of the recombinant production of proteins generally requires the availability of new regulatory sequences which are suitable for controlling the expression of the proteins in a host cell. One such regulatory sequence includes the sequence encoding the yeast alpha-factor signal peptide first disclosed in Kurjan and Herskowitz (Cell, vol. 30: 933-943, 1982). This signal peptide was subsequently shown to be applicable as a general secretion signal for the production of heterologous polypeptides in yeast (EP 0 116 201 B1).

Allison and Young, (Molecular and Cellular Biology, 1989, vol:9(11) p. 4977-4985) describes the effect of mutations in the signal sequence of the alpha factor. All the described mutations resulted in a decrease in alpha factor by 2 to 12 fold.

It is an object of the present invention to provide improved methods for producing a polypeptide in a fungal host cell using derivatives of the yeast alpha-factor signal peptide.

### SUMMARY OF THE INVENTION

The invention provides a method for increased production of a secreted polypeptide, comprising: (a) cultivating a fungal host cell in a medium conducive for the production of the polypeptide, wherein the host cell comprises a nucleic acid construct comprising a first nucleotide sequence encoding a signal peptide operably linked to a second nucleotide sequence encoding the polypeptide, wherein the first nucleotide sequence is foreign to the second nucleotide sequence, the 3′ end of the first nucleotide sequence is upstream of the second nucleotide sequence, and the first nucleotide sequence is selected from the group consisting of a nucleotide sequence encoding an alpha factor signal peptide having at least one substitution of an Alanine in position 9; and (b) isolating the secreted polypeptide from the cultivation medium.

In a second aspect the invention relates to isolated variants of the alpha factor signal peptide, wherein the amino acid in position 9, or the corresponding position as determined by alignment with the wild type sequence, has been modified to include one of the substitutions selected from the group consisting of A9T, A9S, A9H, A9I, A9F, and A9G.

Furthermore, the present invention provides for a nucleic acid construct comprising a first nucleotide sequence encoding a signal peptide operably linked to a second nucleotide sequence encoding a polypeptide, wherein the first nucleotide sequence is foreign to the second nucleotide sequence, and the 3′ end of the first nucleotide sequence is upstream of the second nucleotide sequence, and the first nucleotide sequence is selected from the group consisting of a nucleotide sequence encoding a signal peptide according to the invention.

The present invention also provides expression vectors and host cells comprising said nucleic acid construct.

In a final aspect the present invention relates to a use of an improved signal peptide for producing a polypeptide, wherein the signal peptide is encoded by a first nucleotide sequence and the polypeptide is encoded by a second nucleotide sequence foreign to the first nucleotide sequence, and the 3′ end of the first nucleotide sequence is upstream of the second nucleotide sequence, and wherein the first nucleotide sequence is selected from the group consisting of a nucleotide sequence encoding an alpha factor signal peptide having at least one substitution of an Alanine in position 9.

### DETAILED DESCRIPTION OF THE INVENTION

### Definitions

The term “variant” when used in reference to another polypeptide or nucleotide sequence is in the context of the present invention to be understood as a polypeptide or nucleotide sequence which comprises a substitution, deletion, and/or insertion of one or several amino acids or nucleotides as compared to another polypeptide (i.e. it is a variant of polypeptide/nucleotide sequence it is compared with). In particular the changes may be of minor nature, such as conservative amino acid substitutions or for nucleotide sequence resulting in conservative amino acid substitutions, that do not significantly affect the activity of the polypeptide; or small deletions, typically of one to about 20 amino acids depending on the size of the polypeptide in which the changes are made. In the present case the signal peptide is only 19 amino acids. Accordingly a small deletion is in the range from 1 to 3 amino acids.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

The first nucleotide sequence encodes a signal peptide of the present invention. The term “signal peptide” or “signal peptide sequence” is defined herein as a peptide sequence usually present at the N-terminal end of newly synthesized secretory or membrane polypeptides which directs the polypeptide across or into a cell membrane of the cell (the plasma membrane in prokaryotes and the endoplasmic reticulum membrane in eukaryotes). It is usually subsequently removed. In particular said signal peptide may be capable of directing the polypeptide into a cell's secretory pathway.

An alpha factor signal peptide according to the present invention is the signal peptide originating from yeast and described in Kurjan and Herskowitz (Cell, vol. 30: 933-943). The wild type alpha factor amino acid sequence is shown in SEQ ID NO: 1.

The term “operably linked” is defined herein as a configuration in which a control sequence, e.g., a signal peptide sequence, is appropriately placed at a position relative to a coding sequence such that the control sequence directs the production of a polypeptide encoded by the coding sequence.

The term “coding sequence” is defined herein as a nucleotide sequence which is translated into a polypeptide when placed under the control of the appropriate control sequences. The boundaries of the coding sequence are generally determined by the start codon located at the beginning (5′ end) of the open reading frame and a stop codon located at the 3′ end of the open reading frame. A coding sequence can include, but is not limited to, genomic DNA, cDNA, RNA, semisynthetic, synthetic, and recombinant nucleotide sequences.

The relatedness between two amino acid sequences is described by the parameter “identity”. For purposes of the present invention, the alignment of two amino acid sequences is determined by using the Needle program from the EMBOSS package (http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

The degree of identity between an amino acid sequence of the present invention (“invention sequence”; e.g. such as amino acids 1 to 19 of SEQ ID NO: 2 and a different amino acid sequence (“foreign sequence”) is calculated as the number of exact matches in an alignment of the two sequences, divided by the length of the “invention sequence” or the length of the “foreign sequence”, whichever is the shortest. The result is expressed in percent identity.

An exact match occurs when the “invention sequence” and the “foreign sequence” have identical amino acid residues in the same positions of the overlap (in the alignment example below this is represented by “|”). The length of a sequence is the number of amino acid residues in the sequence (e.g. the length of SEQ ID NO:2 is 19).

In the purely hypothetical alignment example below, the overlap is the amino acid sequence “HTWGER-NL” of Sequence 1; or the amino acid sequence “HGWGEDANL” of Sequence 2. In the example a gap is indicated by a

Hypothetical Alignment Example:

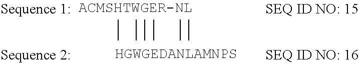

For purposes of the present invention, the degree of identity between two nucleotide sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

In a particular embodiment, the percentage of identity of an amino acid sequence of a polypeptide with, or to, amino acids 1 to 19 of SEQ ID NO: 2 is determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the shortest of the two amino acid sequences, and iv) converting the result of the division of iii) into percentage. The percentage of identity to, or with, other sequences of the invention such as amino acids 1-19 of SEQ ID NO: 3 are calculated in an analogous way.

### Method of the Present Invention

The present invention relates to methods for increased production of a secreted polypeptide, comprising: (a) cultivating a fungal host cell in a medium conducive for the production of the polypeptide, wherein the host cell comprises a nucleic acid construct comprising a first nucleotide sequence encoding a signal peptide operably linked to a second nucleotide sequence encoding the polypeptide, wherein the first nucleotide sequence is foreign to the second nucleotide sequence, the 3′ end of the first nucleotide sequence is upstream of the second nucleotide sequence, and the first nucleotide sequence is selected from the group consisting of a nucleotide sequence encoding an alpha factor signal peptide having at least one substitution of an Alanine in position 9; and (b) isolating the secreted polypeptide from the cultivation medium.

In the methods of the present invention, the fungal host cells are cultivated in a medium conducive for the production of the polypeptide, i.e. in a nutrient medium suitable for production of the polypeptide using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation may take place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection).

The polypeptide may be detected using methods known in the art that are specific for the polypeptide. Such detection methods may include use of specific antibodies, formation of an enzyme product, or disappearance of an enzyme substrate.

In the methods of the present invention, the fungal host cell may in particular produce at least about 25% more, more particularly at least about 50% more, more particularly at least about 75% more, more particularly at least about 100% more, even more particularly at least about 200% more, most particularly at least about 300% more, even more particularly at least about 400% more, even more particularly at least about 500% more, and even most particularly at least about 700% more polypeptide relative to a fungal cell containing a native signal peptide sequence operably linked to a nucleotide sequence encoding the polypeptide when cultured under identical production conditions.

The resulting secreted polypeptide can be recovered directly from the medium by methods known in the art. For example, the polypeptide may be recovered from the nutrient medium by conventional procedures including, but not limited to, centrifugation, filtration, extraction, spray-drying, evaporation, or precipitation.

The polypeptide may be purified by a variety of procedures known in the art including, but not limited to, chromatography (e.g., ion exchange, affinity, hydrophobic, chromato-focusing, and size exclusion), electrophoretic procedures (e.g., preparative isoelectric focusing), differential solubility (e.g., ammonium sulphate precipitation), SDS-PAGE, or extraction (see, e.g., Protein Purification, J.-C. Janson and Lars Ryden, editors, VCH Publishers, New York, 1989).

### Signal Peptide

The 5′ end of the coding sequence of a polypeptide may contain a native nucleotide sequence encoding a signal peptide which is naturally linked with the nucleotide sequence segment which encodes the mature (or pro-form) of the polypeptide. The signal peptide of the present invention may replace the native signal peptide.

In the methods of the present invention, the signal peptide sequence is foreign to the nucleotide sequence encoding a polypeptide of interest, but the signal peptide sequence or nucleotide sequence may be native to the fungal host cell. In this context the term “foreign” is intended to be understood as the signal peptide is not native to the polypeptide, i.e. it originates from another gene than the polypeptide.

In one embodiment the first nucleotide sequence may encode an improved signal peptide having an amino acid sequence which is a variant of SEQ ID NO: 1, and which have the ability to direct a polypeptide into or across a cell membrane (hereinafter “homologous signal peptide”), e.g. into a cell's secretory pathway. SEQ ID NO: 1 shows the normal signal peptide associated with the alpha-factor from the yeast Saccharomyces cerevisiae as described in Kurjan and Herskowitz (Cell vol. 30: 933-943, 1982). In a particular aspect, the signal peptide variants according to the invention may have an amino acid sequence which differs by one amino acid from SEQ ID NO: 1. In particular, the first nucleotide sequence may encode a signal peptide which comprises the amino acid sequence of SEQ ID NO: 1, or a fragment thereof that has the ability to direct the polypeptide into or across a cell membrane, e.g. into a cell's secretory pathway, and wherein the alanine in position 9 (A9A) has been substituted for another amino acid. In a more particular aspect, this substitution at position 9 comprises a substitution selected from the group consisting of A9T, A9S, A9H, A9I, A9F, A9E, A9G. These particular substitutions will result in at least an improvement in expression (measured as secreted product) of a factor 2; in some cases an improvement of up to a factor 8 is observed. The level of improvement will to some degree depend on the polypeptide of interest encoded by the second nucleotide sequence and also by the host cell. Thus in a particular embodiment the invention relates to an isolated signal peptide, selected from the group consisting of: (a) a polypeptide comprising an amino acid sequence having at least 84%, more preferably at least 89%, more preferably at least 94%, identity with SEQ ID NO: 2, and wherein the T in the position corresponding to position 9 of SEQ ID NO: 2 is conserved; and (b) a variant, that retains the ability to direct the polypeptide into or across a cell membrane, comprising a conservative substitution, deletion, and/or insertion of one or several amino acids of amino acids of SEQ ID NO: 2 at a position different from position 9. In another particular embodiment the invention relates to a isolated signal peptide, selected from the group consisting of: (a) a polypeptide comprising an amino acid sequence having at least 84%, more preferably at least 89%, more preferably at least 94%, identity with SEQ ID NO: 3, and wherein the S in the position corresponding to position 9 of SEQ ID NO: 3 is conserved; and (b) a variant, that retains the ability to direct the polypeptide into or across a cell membrane, comprising a conservative substitution, deletion, and/or insertion of one or several amino acids of amino acids of SEQ ID NO: 3 at a position different from position 9. In another particular embodiment the invention relates to a isolated signal peptide, selected from the group consisting of: (a) a polypeptide comprising an amino acid sequence having at least 84%, more preferably at least 89%, more preferably at least 94%, identity with SEQ ID NO: 4, and wherein the H in the position corresponding to position 9 of SEQ ID NO: 4 is conserved; and (b) a variant, that retains the ability to direct the polypeptide into or across a cell membrane, comprising a conservative substitution, deletion, and/or insertion of one or several amino acids of amino acids of SEQ ID NO: 4 at a position different from position 9. In another particular embodiment the invention relates to a isolated signal peptide, selected from the group consisting of: (a) a polypeptide comprising an amino acid sequence having at least 84%, more preferably at least 89%, more preferably at least 94%, identity with SEQ ID NO: 5, and wherein the I in the position corresponding to position 9 of SEQ ID NO: 5 is conserved; and (b) a variant, that retains the ability to direct the polypeptide into or across a cell membrane, comprising a conservative substitution, deletion, and/or insertion of one or several amino acids of amino acids of SEQ ID NO: 5 at a position different from position 9. In another particular embodiment the invention relates to a isolated signal peptide, selected from the group consisting of: (a) a polypeptide comprising an amino acid sequence having at least 84%, more preferably at least 89%, more preferably at least 94%, identity with SEQ ID NO: 6, and wherein the F in the position corresponding to position 9 of SEQ ID NO: 6 is conserved; and (b) a variant, that retains the ability to direct the polypeptide into or across a cell membrane, comprising a conservative substitution, deletion, and/or insertion of one or several amino acids of amino acids of SEQ ID NO: 6 at a position different from position 9. In another particular embodiment the invention relates to a isolated signal peptide, selected from the group consisting of: (a) a polypeptide comprising an amino acid sequence having at least 84%, more preferably at least 89%, more preferably at least 94%, identity with SEQ ID NO: 7, and wherein the E in the position corresponding to position 9 of SEQ ID NO: 7 is conserved; and (b) a variant, that retains the ability to direct the polypeptide into or across a cell membrane, comprising a conservative substitution, deletion, and/or insertion of one or several amino acids of amino acids of SEQ ID NO: 7 at a position different from position 9. In another particular embodiment the invention relates to a isolated signal peptide, selected from the group consisting of: (a) a polypeptide comprising an amino acid sequence having at least 84%, more preferably at least 89%, more preferably at least 94%, identity with SEQ ID NO: 8, and wherein the G in the position corresponding to position 9 of SEQ ID NO: 8 is conserved; and (b) a variant, that retains the ability to direct the polypeptide into or across a cell membrane, comprising a conservative substitution, deletion, and/or insertion of one or several amino acids of amino acids of SEQ ID NO: 8 at a position different from position 9.

In a particular aspect the present invention relates to isolated signal peptides of the invention as specified above in relation to SEQ ID NO: 2, 3, 4, 5, 6, 7, or 8 having an amino acid sequence which has a degree of identity to amino acids 1 to 19 of SEQ ID NO: 2, 3, 4, 5, 6, 7, or 8 of at least 84%, preferably at least 85%, more preferably at least 86%, more preferably at least 87%, more preferably at least 88%, more preferably at least 89%, even more preferably at least 90%, most preferably at least 94%, most preferably at least 95%, most preferably at least 96%, most preferably at least 97%, and even most preferably at least 98%, and which signal peptide retains the ability to direct the polypeptide into or across a cell membrane.

A variant of SEQ ID NO: 1 also comprises fragments, which in the present context means a polypeptide having one or more amino acids deleted from the amino and/or carboxy terminus of this amino acid sequence. In particular a fragment contains at least 10 amino acid residues. such as at least 12 amino acid residues or at least 13 amino acid residues or at least 14 amino acid residues or at least 15 amino acid residues or at least 16 amino residues or at least 17 amino acid residues, or at least 18 amino acid residues. A variant according to the invention could also be a substitution or insertion of one or more amino acids. In such cases where the modified alpha factor signal peptide comprises additional substitutions, insertions or deletions these additional modifications do not affect the ability of the modified alpha factor signal peptide to direct the polypeptide into or across a cell membrane, e.g. into the cell's secretory pathway. The specific modification at position 9 or the position corresponding to position 9 in SEQ ID NO: 1 should however always be present. The actual position in the modified signal peptide will of course depend on the length of the modified signal peptide compared to the wild type and can be found by aligning the modified signal with the wild type signal.

### Polypeptide

The second nucleotide sequence of the present invention encodes a polypeptide of interest. Said polypeptide may be native or heterologous to the fungal host cell in which it is produced.

The term “polypeptide” is not meant herein to refer to a specific length of the encoded product and, therefore, encompasses peptides, oligopeptides, and proteins. Proteins could be the mature form or in the form of a pro-peptide. The term “heterologous polypeptide” is defined herein as a polypeptide which is not native to the fungal cell, a native polypeptide in which modifications have been made to alter the native sequence, or a native polypeptide whose expression is quantitatively altered as a result of a manipulation of the gene encoding the polypeptide by recombinant DNA techniques. The fungal cell may contain one or more copies of the nucleotide sequence encoding the polypeptide.

In particular, the polypeptide may be a hormone or hormone variant, an enzyme, a receptor or fragment thereof, an antibody or fragment thereof, an allergen or a reporter. In a particular aspect, the polypeptide may be an allergen originating from the group of Dermatophagoides sp.

Particularly the Dermatophagoides sp. is selected from the group consisting of Dermatophagoides pteronyssinus, Dermatophagoides farinae, Dermatophagoides siboney, Dermatophagoides microceaus, Blomia tropicalis and Euroglyphus maynei, or an allergen from one of said organisms which subsequently have been modified. More particularly, said allergen may be Der p 1 (SEQ ID NO: 9) or Der p 2 (SEQ ID NO: 10) from Dermatophagoides pteronyssinus. In particular the polypeptide may be the sequence of amino acids 19-320 of SEQ ID NO: 9. In a more particular embodiment the polypeptide may be a variant of the polypeptide sequence of amino acids 19-320 of SEQ ID NO: 9. More particularly said variant may be a S54X or N52X, wherein “X” denotes any amino acid, as said disrupt the N-glycosylation site of Der p 1, in particular said variants may be S54N or N52Q.

In another particular embodiment the polypeptide may be an enzyme, such as an oxidoreductase, transferase, hydrolase, lyase, isomerase, or ligase. In a more particular aspect, the polypeptide may be an aminopeptidase, amylase, carbohydrase, carboxypeptidase, catalase, cellulase, cellobiohydrolase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phospholipase, phytase, polyphenoloxidase, proteolytic enzyme, ribonuclease, transglutaminase, xylanase, or beta-xylosidase. An example of a relevant cellulase includes but is not limited to a cellulase from Mucor cicinellides, e.g. M. cicinellides IFO4554. Examples of relevant proteolytic enzymes include but are not limited to cystein proteases, e.g. cystein protease 5 from Trifolium repens L. An example of a relevant phytase includes but is not limited to a phytase from Peniophora lycii, e.g. P. lycii CBS 686.96.

The second nucleotide sequence encoding a polypeptide of the present invention may be obtained from any prokaryotic, eukaryotic, or other source. For purposes of the present invention, the term “obtained from” as used herein in connection with a given source shall mean that the polypeptide is produced by the source or by a cell in which a gene from the source has been inserted.

The techniques used to isolate or clone a nucleotide sequence encoding a polypeptide of interest are known in the art and include isolation from genomic DNA, preparation from cDNA, or a combination thereof. The cloning of the second nucleotide sequence from such genomic DNA can be effected, e.g., by using the well known polymerase chain reaction (PCR). See, for example, Innis et al., 1990, PCR Protocols: A Guide to Methods and Application, Academic Press, New York. The cloning procedures may involve excision and isolation of a desired nucleotide fragment comprising the nucleotide sequence encoding the polypeptide, insertion of the fragment into a vector molecule, and incorporation of the recombinant vector into the mutant fungal cell where multiple copies or clones of the nucleotide sequence will be replicated. The second nucleotide sequence may be of genomic, cDNA, RNA, semisynthetic, synthetic origin, or any combinations thereof.

In the methods of the present invention, the polypeptide may also be a fused or hybrid polypeptide in which another polypeptide is fused at the N-terminus or the C-terminus of the polypeptide or fragment thereof. A fused polypeptide is produced by fusing a nucleotide sequence (or a fragment thereof) encoding one polypeptide to the second nucleotide sequence (or a fragment thereof) encoding the polypeptide of the present invention. Techniques for producing fusion polypeptides are known in the art, and include, ligating the coding sequences encoding the polypeptides so that they are in frame and expression of the fused polypeptide is under control of the same promoter(s) and terminator. The hybrid polypeptide may comprise a combination of partial or complete polypeptide sequences obtained from at least two different polypeptides wherein one or more may be heterologous to the mutant fungal cell.

Nucleic Acid Construct

The present invention also relates to a nucleic acid construct comprising a first nucleotide sequence encoding a signal peptide of the present invention operably linked to a second nucleotide sequence encoding a polypeptide of the present invention. More particularly the invention relates to a nucleic acid construct comprising a first nucleotide sequence encoding a signal peptide operably linked to a second nucleotide sequence encoding a polypeptide, wherein the first nucleotide sequence is foreign to the second nucleotide sequence, and the 3′ end of the first nucleotide sequence is upstream of the second nucleotide sequence, and the first nucleotide sequence is selected from the group consisting of a nucleotide sequence encoding a signal peptide according to the invention.

“Nucleic acid construct” is defined herein as a nucleotide molecule, either single- or double-stranded, which is isolated from a naturally occurring gene or which has been modified to contain segments of nucleic acids combined and juxtaposed in a manner that would not otherwise exist in nature. The term nucleic acid construct is synonymous with the term expression cassette when the nucleic acid construct contains a coding sequence and all the control sequences required for expression of the coding sequence.

A second nucleotide sequence encoding a polypeptide may be further manipulated in a variety of ways to provide for expression of the polypeptide. Manipulation of the nucleotide sequence prior to its insertion into a vector may be desirable or necessary depending on the expression vector. The techniques for modifying nucleotide sequences utilizing recombinant DNA methods are well known in the art.

In the methods of the present invention, the nucleic acid construct may comprise one or more native control sequences or one or more of the native control sequences may be replaced with one or more control sequences foreign to the first and/or second nucleotide sequence of the nucleic acid construct for improving expression of the second nucleotide sequence encoding a polypeptide in a host cell.

The term “control sequences” is defined herein to include all components which are necessary or advantageous for the expression of the polypeptide encoded by the second nucleotide sequence. Each control sequence may be native or foreign to the second nucleotide sequence encoding the polypeptide. Such control sequences include, but are not limited to, a leader, polyadenylation sequence, propeptide sequence, signal peptide sequence of the present invention, and transcription terminator. At a minimum, the control sequences include a signal peptide sequence of the present invention, and transcriptional and translational stop signals. The control sequences may be provided with linkers for the purpose of introducing specific restriction sites facilitating ligation of the control sequences with the second nucleotide sequence encoding the polypeptide.

The control sequence may be an appropriate promoter sequence, which is recognized by a host cell for expression of the nucleotide sequence. The promoter sequence contains transcriptional control sequences which mediate the expression of the polypeptide. The promoter may be any sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase, Fusarium oxysporum trypsin-like protease (WO 96/00787), Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

In a yeast host, useful promoters include but are not limited to those obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1,ADH2/GAP), Saccharomyces cerevisiae triose phosphate isomerase (TPI), Saccharomyces cerevisiae metallothionine (CUP1), and Saccharomyces cerevisiae 3-phosphoglycerate kinase. Other useful promoters for yeast host cells are described by Romanos et al., 1992, Yeast 8: 423-488.

The control sequence may be a suitable transcription terminator sequence, which is recognized by a host cell to terminate transcription. The terminator sequence is operably linked to the 3′ terminus of the nucleotide sequence encoding the polypeptide. Any terminator which is functional in the host cell of choice may be used in the present invention.

Examples of suitable terminators for filamentous fungal host cells include but are not limited to those obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Examples of suitable terminators for yeast host cells include but are not limited to those obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a polyadenylation sequence, which is operably linked to the 3′ end of the nucleotide sequence encoding a polypeptide and which, when transcribed, is recognized by the host cell as a signal to add polyadenosine residues to transcribed mRNA. Any polyadenylation sequence which is functional in the host cell of choice may be used in the present invention.

Examples of suitable polyadenylation sequences for filamentous fungal host cells include but are not limited to those obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells include but are not limited to those described by Guo and Sherman, 1995, Molecular Cellular Biology 15: 5983-5990.

The control sequence may also be a propeptide coding region that codes for an amino acid sequence positioned at the amino terminus of a polypeptide. The resultant polypeptide is known as a proenzyme or propolypeptide (or a zymogen in some cases). A pro-polypeptide is generally inactive and can be converted to a mature active polypeptide by catalytic or autocatalytic cleavage of the propeptide from the propolypeptide. Examples of propeptide coding region include but are not limited to those obtained from the genes for Dermantophagoides pteronyssinus Der p 1, or other genes obtained from Dermantophagoides, Fusarium oxysporum trypsin, Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region. If a propeptide is present a cleavable site may in one embodiment be present between the propeptide and the mature polypeptide. The term “cleavable site” is to be understood as an amino acid sequence which is recognized by a proteolytic enzyme capable of cleaving the polypeptide at this site. Examples of such site include a kex-site, in particular a kex-II site.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleotide sequence encoding the polypeptide would be operably linked with the regulatory sequence.

Expression Vector

The present invention also relates to a recombinant expression vector comprising a nucleic acid construct of the present invention. Besides comprising a first and a second nucleotide sequence encoding a signal peptide and a polypeptide, respectively of the present invention said expression vector may in particular comprise a transcriptional and translational stop signal. The various nucleotide and control sequences described above may be joined together to produce a recombinant expression vector which may include one or more convenient restriction sites to allow for insertion or substitution of the promoter and/or nucleotide sequence encoding the polypeptide at such sites. Alternatively, the nucleic acid construct may be inserted into an appropriate vector for expression for expression of the polypeptide encoded by the second nucleotide sequence. In creating the expression vector, the second nucleotide sequence encoding the polypeptide is located in the vector so that said sequence is operably linked with a signal peptide sequence of the present invention and one or more appropriate control sequences for expression.

The recombinant expression vector may be any vector (e.g., a plasmid or virus) which can be conveniently subjected to recombinant DNA procedures and can bring about the expression of the nucleotide sequence. The choice of the vector will typically depend on the compatibility of the vector with the host cell into which the vector is to be introduced. The vectors may be linear or closed circular plasmids.

The vectors of the present invention may in particular contain one or more selectable markers which permit easy selection of transformed cells. A selectable marker is a gene the product of which provides for biocide or viral resistance, resistance to heavy metals, prototrophy to auxotrophs, and the like. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents thereof. In particular for use in an Aspergillus cell are the amdS and pyrG genes of Aspergillus nidulans or Aspergillus oryzae and the bar gene of Streptomyces hygroscopicus.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention may in particular contain an element(s) that permits stable integration of the vector into the host cell's genome or autonomous replication of the vector in the cell independent of the genome.

For integration into the host cell genome, the vector may rely on the second nucleotide sequence encoding the polypeptide or any other element of the vector for stable integration of the vector into the genome by homologous or nonhomologous recombination. Alternatively, the vector may contain additional nucleotide sequences for directing integration by homologous recombination into the genome of the host cell. The additional nucleotide sequences may enable the vector to be integrated into the host cell genome at a precise location(s) in the chromosome(s). To increase the likelihood of integration at a precise location, the integrational elements should in particular contain a sufficient number of nucleotides, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication which functions in a cell. The term “origin of replication” or “plasmid replicator” is defined herein as a nucleotide sequence that enables a plasmid or vector to replicate in vivo.

Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its ability to function temperature-sensitive in the host cell (see, e.g., Ehrlich, 1978, Proceedings of the National Academy of Sciences USA 75: 1433).

Examples of origins of replication useful in a filamentous fungal host cell are AMA1 and ANS1 (Gems et al., 1991, Gene 98:61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

More than one copy of a nucleotide sequence encoding a polypeptide may be inserted into the host cell to increase production of the gene product. An increase in the copy number of the nucleotide sequence can be obtained by integrating at least one additional copy of the sequence into the host cell genome or by including an amplifiable selectable marker gene with the nucleotide sequence where cells containing amplified copies of the selectable marker gene, and thereby additional copies of the nucleotide sequence, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention relates to methods in which polypeptides are produced in a fungal host cell and to a recombinant host cells comprising a nucleic acid construct of the present invention.

A vector comprising a first nucleotide sequence encoding a signal peptide of the present invention operably linked to a second nucleotide sequence encoding a polypeptide is introduced into a fungal host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any fungal cell useful in the methods of the present invention. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a particular aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No.9,1980).

In a more particular aspect, the yeast host cell is a Candida, Hansenula, Kluyveromyces, Pichia, Saccharomyces, Schizosaccharomyces, or Yarrowia cell.

In a most particular aspect, the yeast host cell is a Saccharomyces carlsbergensis, Saccharomyces cerevisiae, e.g S. cerevisiae YNG318, Saccharomyces diastaticus, Saccharomyces douglasii, Saccharomyces kluyveri, Saccharomyces norbensis, Saccharomyces oviformis, Kluyveromyces lactis, Pichia pastoris or Yarrowia lipolytica cell.

In another particular aspect, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In a more particular aspect, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, or Trichoderma.

In an even more particular aspect, the filamentous fungal host cell is an Aspergillus awamori, Aspergillus foetidus, Aspergillus japonicus, Aspergillus nidulans, Aspergillus niger or Aspergillus oryzae cell. In another even more particular aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another even more particular aspect, the filamentous fungal host cell is a Humicola insolens, Humicola lanuginosa, Mucor miehei, Myceliophthora thermophila, Neurospora crassa, Penicillium purpurogenum, Thielavia terrestris, Trichoderma harzianum, Trichoderma koningii, Trichoderma longibrachiatum, Trichoderma reesei, or Trichoderma viride cell.

In a most particular aspect, the Fusarium venenatum cell is Fusarium venenatum A3/5, which was originally deposited as Fusarium graminearum ATCC 20334 and recently reclassified as Fusarium venenatum by Yoder and Christianson, 1998, Fungal Genetics and Biology 23: 62-80 and O'Donnell et al., 1998, Fungal Genetics and Biology 23: 57-67; as well as taxonomic equivalents of Fusarium venenatum regardless of the species name by which they are currently known. In another particular aspect, the Fusarium venenatum cell is a morphological mutant of Fusarium venenatum A3/5 or Fusarium venenatum ATCC 20334, as disclosed in WO 97/26330.

In another most particular aspect, the Trichoderma cell is Trichoderma reesei ATCC 56765, Trichoderma reesei ATCC 13631, Trichoderma reesei CBS 526.94, Trichoderma reesei CBS 529.94, Trichoderma longibrachiatum CBS 528.94, Trichoderma longibrachiatum ATCC 2106, Trichoderma longibrachiatum CBS 592.94, Trichoderma viride NRRL 3652, Trichoderma viride CBS 517.94, or Trichoderma viride NIBH FERM/BP 447.

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable procedures for transformation of Trichoderma reesei host cells are described in Penttila et al., 1987, Gene 61: 155-164, and Gruber et al., 1990, Curr Genet. 18(1):71-6. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Materials and Methods

### Strains and Plasmids

### Strains

E. coli DH12S (available from Gibco BRL) is used for yeast plasmid rescue. Saccharomyces cerevisiae YNG318: MATa Dpep4[cir+] ura3-52, leu2-D2, his 4-539 is described in J. Biol. Chem. 272 (15), 9720-9727, 1997).

### Plasmids

All yeast expression vectors are S. cerevisiae and E. coli shuttle vectors under the control of TPI promoter, constructed from pJC039 described in WO 00/10038.

### Genes

Der p 1 from Dermatophagoides pteronyssinus: NCBI accession number: P08176, the amino acid sequence is shown in SEQ ID NO: 9

Der p 2 from Dermatophagoides pteronyssinus: NCBI accession number: P49278, the amino acid sequence is shown in SEQ ID NO: 10

### Medium and Substrates

RS-25: 40 g/L soy bean powder, 40 g/L glucose, 10 g/L KH₂PO₄, 0.25 g/L MgSO₄, 0.01 g/L FeSO₄, 2.5 g/L NH₄NO₃; pH 6
YPD: 20 g/L Glucose, 20 g/L Pepton and 10 g/L Yeast extract
10×Basal solution: 66.8 g/l Yeast nitrogen base w/o amino acids (DI FCO), 100 g/l succinate and 60 g/l NaOH.
SC-glucose (or SC-medium): 100 ml/l 20% glucose (i.e., a final concentration of 2%=2 g/100 ml), 4 ml/l 5% threonine, 10 ml/l 1% tryptophan, 25 ml/l 20% casamino acids and 100 ml/l 10 ×basal solution. This solution was sterilized using a filter of a pore size of 0.20 Microm. Agar and H₂O (approx. 761 ml) is autoclaved together, and the separately sterilized SC-glucose solution is added to the agar solution.
PEG/LiAc solution: 50 ml 40% PEG4000 (sterilized by autoclaving) and 1 ml 5M Lithium Acetate (sterilized by autoclaving).

### Methods

### Yeast Transformation

This method was used in examples 1 and 2.

To transform yeast the in vivo recombinant mechanism was utilised by which it is possible for yeast to recombine a vector sequence and PCR fragments in vivo to create an expression vector, if both the vector sequence and the PCR fragments have the same flanking regions.

A DNA mixture was prepared by mixing 0.5 MicroL of vector (EcoRI-NotI digested) and 1 MicroL of PCR fragments. S. cerevisiae YNG318 competent cells were thawed on ice. One hundred MicroL of the cells were mixed with the DNA mixture and 10 MicroL of carrier DNA (Clontech) in 12 ml polypropylene tubes (Falcon 2059). To this 0.6ml PEG/LiAc solution was added and mixed gently and then incubated for 30 min at 30° C., and 200 rpm. Thereafter it was incubated for 30 min at 42° C. (heat shock) before transferring it to an eppendorf tube and centrifugation for 5 sec. The supernatant was removed and resolved in 3 ml of YPD. The cell suspension was then incubated for 45 min at 200 rpm at 30° C. before it was poured on to SC-glucose plates.

### PCR Reaction

Unless otherwise indicated the PCR reactions were carried out under the following conditions:

The PCR reaction contained 38.9 MicroL H₂O, 5 MicroL 10 × reaction buffer, 1 MicroL Klen Taq LA (Clontech), 4 MicroL 10 mM dNTPs, 0.3 MicroL×2 100 pmol/MicroL primer and 0.5 MicroL template DNA and was carried out under the following conditions: 30 cycles of 10 sec at 98° C. and 90 sec at 68° C., and a final 10 min at 68° C.

### Sandwich ELISA

Immunoplates (Nunc Maxisorb; Nunc-Nalgene) were coated overnight at 4° C. with a suitable dose of polyclonal rabbit anti Der p 1 or Der p 2 immunoglobin for Der p 1 or Der p 2 determinations, respectively. The plates were then washed thoroughly with 0.15 M Phosphate Buffered Saline (PBS) containing 0.05% Tween 20 (PBST), and remaining binding sites are blocked with PBS with 2% skim milk powder, 1 h at room temperature. Samples, it can be purified, semi-purified recombinant mite polypeptide allergen or crude culture broth containing protein of interest, were added in a suitable dose or dose-range. The plates were then washed thoroughly with 0.15 M PBST before the allergens were detected by incubation with biotinylated anti Der p 1 or Der p 2 antibody, respectively, 1 h at room temperature. The plates were then washed again in 0.15 M PBST before conjugated with complexes of Streptavidin:Horse Radish Peroxidase (Kierkegaard & Perry) for 1 h at room temperature. The washing step was repeated and then the plates were developed by adding 3,3′,5,5′-tetramethylbenzidine hydrogen peroxide (TMB Plus, Kem-En-Tec) before the reaction was stopped by addition of 0.2 M H₂SO₄. The optical density (OD) at 450 nm reflected allergen binding to the immunoglobin, and it was then possible to detect and also determine the amount of allergen bound by comparing with the data obtained for natural Der p 1 or Der p 2 (available from InDoor biotechnologies) which were included in the ELISA in a known concentration dose range.

### Other Methods

E. coli transformation to rescue yeast plasmid was carried out by electroporation (BIO-RAD Gene Pulser).

DNA Plasmids were prepared with the Qiagen® Plasmid Kit. DNA fragments and recovered from agarose gel by the Qiagen gel extraction Kit.

PCR was carried out by the PTC-200 DNA Engine.

The ABI PRISMTM 310 Genetic Analyzer was used for determination of all DNA sequences. Yeast total DNA was extracted by the Robzyk and Kassir's method described in Nucleic acids research vol. 20, No. 14 (1992) 3790.

### Examples

### Example 1

Expression of Der p 1 in S. cerevisiae

Expression levels of a polypeptide of interest using the improved alpha factor signal of the invention can be illustrated by expression levels of e.g. Der p1. Any expression vector suitable for expression of polypeptides in yeast can be used. In the description below an example of a suitable expression construct based on the yeast pYES2 vector is described. From genomic DNA of S. cerevisiae the TPI-promoter is amplified by a standard PCR-reaction using PWO-polymerase.

### Primers:

(SEQ ID NO: 11)

Forward primer:
5′TACTGACACCGGTGCATGCCTGCAGGTCGACTCTAG 3′

(SEQ ID NO: 12)

Reverse primer:
5′TAAGTAAAGCTTTCTTTTAATCGTTTATATTGTGTATG 3′

The amplified product is purified over a Qiaquick Spin Column. The purified product and the vector pYes2 are digested with HinDIII/AgeI in Nebuffer 2 (New England Biolabs). This digestion removes the Gal1-promotor from the vector. The vector and insert fragment are purified from a 1.5% preparative agarose gel using the QIAquick method (Qiagen). Vector and inserts are ligated by T4-DNAligase, transformed into CaCl₂-competente DH10B-cells and plated onto LB plates containing ampicillin (100 μg/ml). The cloning is verified by DNA sequencing and named pYES-TPI.

Oligos for introduction of the alpha signal with the amino acid sequence MRFPSIFTAVLFAASSALA, and for amplification of Der p 1 are designed: A forward DNA oligo has from the 5′ end sequence overlap to pYES-TPI sequence upstream of the HinDIII site of the plasmid (1-42), and with sequence encoding the alpha signal (43-99) and with overlapping sequence to the Der p 1 gene encoding the N-terminus of the propeptide (100-132):

(SEQ ID NO: 13)

5′GAGTGTTGTTCATACACAATATAAACGATTAAAAGAAAGCTTATGAGA
TTTCCTTCTATTTTTACTGCTGTTTTATTCGCTGCTTCCTCCGCTTTAGC
TCGCCCGAGCAGCATTAAAACCTTCGAAGAATAT 3′

A reverse oligo has from the 5′ end sequence overlap to pYES-TPI downstream of the HinDIII site of the plasmid (1-40) and to the C-terminus encoding end of the Der p 1 gene (41-68):

(SEQ ID NO: 14)

5′ACTAATTACATGATGCGGCCCTCTAGATCTCGACTCATTACAGGATAA
CAACGTACGGGTATTCTTCG 3′

The alpha-signal-proDer p 1 gene is PCR amplified by standard reactions and DNA purified.

The pYES-TPI expression vector is digested with HinDIII (New England Biolabs) and the linearized vector is purified and used for transformation of S. cerevisiae together with the alpha-signal-proDer p 1 amplified gene (Sambrook et al., Molecular Cloning: A Laboratory Manual, 2^{nd} Ed., Cold Spring Harbor, 1989). The vector fragment and the amplified gene recombined in yeast cells by gap repair (Orr-Weaver and Szostak, PNAS, 1983, vol. 80, p. 4417-4421). After transformation the transformants are plated on SC agar containing 2% glucose. After incubation at 30° C. for three to four days, the colonies are grown in SC medium 2% glucose for plasmid rescue and DNA sequencing of the alpha-signal-pro Der p 1 gene.

Alpha signal variant is constructed in the same way except for the forward primer where position 67-69 encoding Ala in position 9 from the Met are changed to a codon usage encoding Thr or any of the other relevant substitutions.

Other expression constructs may also be applied. In one set of data the pSteD212 vector was used, which is derived from yeast expression vector pYES 2.0 (Invitrogen, UK and Kofod et al. 1994 J. Biol. Chem. 269: 29182-29189). This plasmid replicated both in E. coli and in S. cerevisiae. In S. cerevisiae Der p 1 was expressed from this plasmid.

pSteD212 is an episomal expression vector containing URA3, gene of the synthetic pathway for uracil, encoding oritidine 5′-decarboxylase which allows for selection on minimal medium. The vector further contains 2 my Origin, origin of DNA replication to ensure multicopy of the plasmid in both yeast and E. coli. The TPI (triose-phosphate isomerase) promoter ensures constitutive expression of the gene of interest which can be cloned into a multiple cloning site (mcs) placed downstream of the promoter. A yeast transcriptional terminator is present downstream of the mcs. The ampicillin resistance gene also carried on pSteD212 is used for selection in E. coli. Thus the pSteD212 vector is functionally and structurally very similar to pYES2.

A more detailed description on the elements described above for gene expression vectors can be found in Romanos et al., 1992, Yeast, 8, 423-488 and Sambrook et al. (Molecular Cloning: A Laboratory Manual, 2nd Ed., Cold Spring Harbor, 1989).

Recombinant Der p 1 was expressed with the Der p 1 propeptide and had the mutation S54N which disrupts the only N-glycosylation motif within the mature sequence.

For screening of yeast transformants expressing Der p 1, the transformation solution was plated on SC-agar plates for colony formation at 30° C. for 3 days. Colonies were inoculated in 50 ml sterile plastic tubes, each tube containing 10 mL SC medium. The tubes were fermented in 500 ml baffled Erlenmeyer flasks containing 100 ml SC medium at 30° C., 250 rpm for 4 days. Culture broth from these fermentations were used for sandwich ELISA experiments to determine the concentration of expressed protein.

The expression level of Der p 1 S54N either expressed by the wild type alpha signal or by the A9T alpha signal was determined by sandwich ELISA as described above in the Method section. Expression of Der p 1 S54N with the A9T alpha signal was increased about 5 times compared to expression of same protein with the wild type alpha signal.

### Example 2

Expression of Der p 2 in S. cerevisiae

As another illustrative example according to the invention expression of Der p2 using different variants of the signal peptide according to the invention is described.

In the description below an example of a suitable expression construct based on the yeast pYES2 vector is described.

Der p 2 from Dermatophagoides pteronyssinus (the amino acid sequence of which is depicted in SEQ ID NO: 10) encoding gene is located in vector pYES-TPI, which is derived from yeast expression vector pYES2 (Invitrogen) as described in example 1.

This plasmid replicates both in E. coli and in S. cerevisiae. In S. cerevisiae Der p 2 can be expressed from this plasmid.

One set of experimental data was obtained using pSteD212 as the expression vector.

For secretion in yeast different alpha signal peptide variants were tested for their expression efficiency by introducing them upstream of the encoding Der p 2 gene. One alpha signal was the wild type signal with the amino acid sequence MRFPSIFTAVLFAASSALA and the other signals were the same except for amino acid changes in position A9. The Alanine in position 9 was substituted to S, H, I, F, E, G or T by standard oligo and PCR technology as described in example 1, but with primers with overlap to the Der p 2 sequence. Expression constructs with the different signal peptides were made by cloning of DNA fragments also described above. The constructs were transformed into S. cerevisiae. For screening of yeast transformants expressing Der p 2, the transformation solution was plated on SC-agar plates for colony formation at 30° C. for 3 days. Colonies were inoculated in 50 ml sterile plastic tubes, each tube containing 10 mL SC medium. The tubes were fermented in 500 ml baffled Erlenmeyer flasks containing 100 ml SC medium at 30° C., 250 rpm for 4 days. Culture broth from these fermentations was used for sandwich ELISA experiments to determine the concentration of expressed protein as described above.

The expression level of Der p 2 by the different alpha signal mutants were determined by sandwich ELISA and compared to Der p 2 expression with wild type alpha signal (A9A). Times improved expression compared to wild type alpha signal (A9A) are given in the table below:

| | Times expression of Der |
|---|---|
| | p 2 compared to wild |
| Alpha leader substitution | type signal (A9A). |
| MRFPSIFTAVLFAASSALA (A9A) | 1 X |
| (SEQ ID NO: 1) | |
| MRFPSIFTTVLFAASSALA (A9T) | 7 X |
| (SEQ ID NO: 2) | |
| MRFPSIFTSVLFAASSALA (A9S) | 7 X |
| (SEQ ID NO: 3) | |
| MRFPSIFTHVLFAASSALA (A9H) | 7 X |
| (SEQ ID NO: 4) | |
| MRFPSIFTIVLFAASSALA (A9I) | 8 X |
| (SEQ ID NO: 5) | |
| MRFPSIFTFVLFAASSALA (A9F) | 5 X |
| (SEQ ID NO: 6) | |
| MRFPSIFTEVLFAASSALA (A9E) | 2 X |
| (SEQ ID NO: 7) | |
| MRFPSIFTGVLFAASSALA (A9G) | 3 X |
| (SEQ ID NO: 8) | |

1. A method for increased production of a secreted polypeptide, comprising:
(a) cultivating a fungal host cell in a medium conducive for the production of the polypeptide, wherein the host cell comprises a nucleic acid construct comprising a first nucleotide sequence encoding a signal peptide operably linked to a second nucleotide sequence encoding the polypeptide, wherein the first nucleotide sequence is foreign to the second nucleotide sequence, the 3′ end of the first nucleotide sequence is upstream of the second nucleotide sequence, and the first nucleotide sequence is selected from the group consisting of a nucleotide sequence encoding an alpha factor signal peptide having at least one substitution of an Alanine in position 9; and
(b) isolating the secreted polypeptide from the cultivation medium.

2. A method in accordance with claim 1, wherein the at least one modification in the amino acid sequence encoded by the first nucleotide sequence, A9Xaa, is selected from the group consisting of A9T, A9S, A9H, A9I, A9F, A9E, and A9G.

3. A method in accordance with claim 1, wherein the modified alpha factor signal peptide comprises additional substitutions or deletions which do not affect the ability of the modified alpha factor signal peptide to direct the polypeptide into or across a cell membrane, e.g. into the cell's secretory pathway.

4. A method in accordance with claim 1, wherein the second nucleotide sequence encodes a polypeptide native to the fungal host cell.

5. A method in accordance with claim 1, wherein the second nucleotide sequence encodes a polypeptide heterologous to the fungal host cell.

6. A method in accordance with claim 1, wherein the fungal host cell contains more than one copy of the first and the second nucleotide sequences.

7. A method in accordance with claim 1, wherein the second nucleotide sequence encodes a hormone or hormone variant, an enzyme, receptor or fragment thereof, an antibody or fragment thereof, an allergen or reporter.

8. A method accordance with claim 7, wherein the enzyme is an oxidoreductase, transferase, hydrolase, lyase, isomerase, or ligase.

9. A method in accordance with claim 8, wherein the enzyme is an aminopeptidase, amylase, carbohydrase, carboxypeptidase, catalase, cellulase, cellobiohydrolase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phospholipase, phytase, polyphenoloxidase, proteolytic enzyme, ribonuclease, transglutaminase, xylanase, or beta-xylosidase.

10. A method in accordance with claim 7, wherein the second nucleotide sequence encodes an allergen from the group of Dermatophagoides sp.

11. A method in accordance with claim 10, wherein the Dermatophagoides sp. is selected from the group consisting of Dermatophagoides pteronyssinus, Dermatophagoides farinae, Dermatophagoides siboney, Dermatophagoides microceaus.

12. A method in accordance with claim 11, wherein the allergen is selected from the group consisting of Der p 1 or Der p 2 from Dermatophagoides pteronyssinus, or variants thereof.

13. A method in accordance with claim 1, wherein the fungal host cell is a filamentous fungal host cell.

14. A method according to claim 13, wherein the filamentous fungal host cell is selected from the group consisting of Acremonium, Aspergillus, Fusarium, Humicola, Mucor, Myceliophthora, Neurospora, Penicillium, Thielavia, Tolypocladium, or Trichoderma.

15. A method according to claim 14, wherein the Aspergillus host cell is selected from the group consisting of Aspergillus awamori, Aspergillus foetidus, Aspergillus japonicus, Aspergillus nidulans, Aspergillus niger or Aspergillus oryzae

16. A method in accordance with claim 1, wherein the fungal host cell is a yeast cell.

17. A method according to claim 16, wherein the yeast cell is a Saccharomyces cell or a Pichia cell.

18. A method according to claim 17, wherein the yeast cell is a S. cerevisiae cell or a P. pastoris cell

19. An isolated signal peptide, selected from the group consisting of: (a) a polypeptide comprising an amino acid sequence having at least 84%, more preferably at least 89%, more preferably at least 94%, identity to SEQ ID NO: 2, and wherein the T in the position corresponding to position 9 of SEQ ID NO: 2 is conserved; and (b) a variant, that retains the ability to direct the polypeptide into or across a cell membrane, comprising a conservative substitution, deletion, and/or insertion of one or several amino acids of amino acids of SEQ ID NO: 2 at a position different from position 9.

20. An isolated signal peptide, selected from the group consisting of: (a) a polypeptide comprising an amino acid sequence having at least 84%, more preferably at least 89%, more preferably at least 94%, identity with SEQ ID NO: 3, and wherein the S in the position corresponding to position 9 of SEQ ID NO: 3 is conserved; and (b) a variant, that retains the ability to direct the polypeptide into or across a cell membrane, comprising a conservative substitution, deletion, and/or insertion of one or several amino acids of amino acids of SEQ ID NO: 3 at a position different from position 9.

21. An isolated signal peptide, selected from the group consisting of: (a) a polypeptide comprising an amino acid sequence having at least 84%, more preferably at least 89%, more preferably at least 94%, identity with SEQ ID NO: 4, and wherein the H in the position corresponding to position 9 of SEQ ID NO: 4 is conserved; and (b) a variant, that retains the ability to direct the polypeptide into or across a cell membrane, comprising a conservative substitution, deletion, and/or insertion of one or several amino acids of amino acids of SEQ ID NO: 4 at a position different from position 9.

22. An isolated signal peptide, selected from the group consisting of: (a) a polypeptide comprising an amino acid sequence having at least 84%, more preferably at least 89%, more preferably at least 94%, identity with SEQ ID NO: 5, and wherein the I in the position corresponding to position 9 of SEQ ID NO: 5 is conserved; and (b) a variant, that retains the ability to direct the polypeptide into or across a cell membrane, comprising a conservative substitution, deletion, and/or insertion of one or several amino acids of amino acids of SEQ ID NO: 5 at a position different from position 9.

23. An isolated signal peptide, selected from the group consisting of: (a) a polypeptide comprising an amino acid sequence having at least 84%, more preferably at least 89%, more preferably at least 94%, identity with SEQ ID NO: 6, and wherein the F in the position corresponding to position 9 of SEQ ID NO: 6 is conserved; and (b) a variant, that retains the ability to direct the polypeptide into or across a cell membrane, comprising a conservative substitution, deletion, and/or insertion of one or several amino acids of amino acids of SEQ ID NO: 6 at a position different from position 9.

24. An isolated signal peptide, selected from the group consisting of: (a) a polypeptide comprising an amino acid sequence having at least 84%, more preferably at least 89%, more preferably at least 94%, identity with SEQ ID NO: 8, and wherein the G in the position corresponding to position 9 of SEQ ID NO: 8 is conserved; and (b) a variant, that retains the ability to direct the polypeptide into or across a cell membrane, comprising a conservative substitution, deletion, and/or insertion of one or several amino acids of amino acids of SEQ ID NO: 8 at a position different from position 9.

25. A nucleic acid construct comprising a first nucleotide sequence encoding a signal peptide operably linked to a second nucleotide sequence encoding a polypeptide, wherein the first nucleotide sequence is foreign to the second nucleotide sequence, and the 3′ end of the first nucleotide sequence is upstream of the second nucleotide sequence, and the first nucleotide sequence is selected from the group consisting of a nucleotide sequence encoding a signal peptide in accordance with claim 19.

26. A recombinant expression vector comprising the nucleic acid construct of claim 25.

27. A recombinant host cell comprising the nucleic acid construct of claim 25.

28. Use of an improved signal peptide for producing a polypeptide, wherein the signal peptide is encoded by a first nucleotide sequence and the polypeptide is encoded by a second nucleotide sequence foreign to the first nucleotide sequence, and the 3′ end of the first nucleotide sequence is upstream of the second nucleotide sequence, and wherein the first nucleotide sequence is selected from the group consisting of a nucleotide sequence encoding an alpha factor signal peptide having at least one substitution of an Alanine in position 9.


SEQUENCE LISTING (16 sequences)

SEQ ID NO: 1 - protein, 19 aa, Saccharomyces cerevisiae
MRFPSIFTAVLFAASSALA

SEQ ID NO: 2 - protein, 19 aa, Artificial
  alpha factor signal variant
MRFPSIFTTVLFAASSALA

SEQ ID NO: 3 - protein, 19 aa, Artificial
  alpha factor signal variant
MRFPSIFTSVLFAASSALA

SEQ ID NO: 4 - protein, 19 aa, Artificial
  alpha factor signal variant
MRFPSIFTHVLFAASSALA

SEQ ID NO: 5 - protein, 19 aa, Artificial
  alpha factor signal variant
MRFPSIFTIVLFAASSALA

SEQ ID NO: 6 - protein, 19 aa, Artificial
  alpha factor signal variant
MRFPSIFTFVLFAASSALA

SEQ ID NO: 7 - protein, 19 aa, Artificial
  alpha factor signal variant
MRFPSIFTEVLFAASSALA

SEQ ID NO: 8 - protein, 19 aa, Artificial
  alpha factor signal variant
MRFPSIFTGVLFAASSALA

SEQ ID NO: 9 - protein, 302 aa, Dermatophagoides pteronyssinus
  propeptide (1)..(80)
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQNLDLAEQELVDCASQHGCHGDTIPRGIEYIQHNGVVQESYYRYVAREQSCRRPNAQRFGISNYCQIYPPNANKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 10 - protein, 129 aa, Dermatophagoides pteronyssinus
DQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 11 - DNA, 36 bp, Artificial
  PCR primer
tactgacaccggtgcatgcctgcaggtcgactctag

SEQ ID NO: 12 - DNA, 38 bp, Artificial
  PCR primer
taagtaaagctttcttttaatcgtttatattgtgtatg

SEQ ID NO: 13 - DNA, 132 bp, Artificial
  PCR primer
gagtgttgttcatacacaatataaacgattaaaagaaagcttatgagatttccttctatttttactgctgttttattcgctgcttcctccgctttagctcgcccgagcagcattaaaaccttcgaagaatat

SEQ ID NO: 14 - DNA, 68 bp, Artificial
  PCR primer
actaattacatgatgcggccctctagatctcgactcattacaggataacaacgtacgggtattcttcg

SEQ ID NO: 15 - protein, 12 aa, Artificial
  alignment example seq 1
ACMSHTWGERNL

SEQ ID NO: 16 - protein, 14 aa, Artificial
  alignment example seq 2
HGWGEDANLAMNPS